(19) United States
(12) Patent Application Publication
(10) Pub. No.: US 2020/0138734 A1
(43) Pub. Date: May 7, 2020

(54) TOPICAL FORMULATION FOR TREATING BRUISING

(71) Applicant: Jerry McSHANE, Deer Park, TX (US)

(72) Inventor: Jerry McSHANE, Deer Park, TX (US)

(21) Appl. No.: 16/728,329

(22) Filed: Dec. 27, 2019

Related U.S. Application Data
(63) Continuation-in-part of application No. 16/042,966, filed on Jul. 23, 2018.

(51) Int. Cl. A61K 31/045; A61K 47/22; A61K 47/10; A61K 47/34; A61K 33/14; A61K 9/00; A61P 7/02

(52) U.S. Cl. CPC A61K 31/045; A61K 47/22; A61K 47/10; A61P 7/02; A61K 33/14; A61K 9/0014; A61K 47/34

(57) ABSTRACT

A topical formulation for treating bruising can include a protein denaturant, preferably 2-butanol, a penetration enhancer, preferably a combination of dimethyl isosorbide and propylene glycol, and water. The topical formulation can be particularly effective for reversing bruising caused by Senile Purpura and/or preventing hemosiderin staining.

### CROSS REFERENCE TO RELATED APPLICATIONS

This application is a continuation-in-part of Ser. No. 16/042,966, filed Jul. 23, 2018, now pending.

### BACKGROUND

### 1. Field

The disclosure of the present patent application relates to topical compositions, and particularly to a topical formulation for treating bruising.

### 2. Description of the Related Art

Senile Purpura, sometimes referred to as Batman's Purpura, is a dark blue-black discoloration of the skin caused by either spontaneous or post-traumatic bleeding of, most commonly, the upper extremities and shoulders. Although the size can vary, these discolorations are usually less than five centimeters in diameter and have well demarcated smooth edges. The underlying cause can be traced to the increased friability of the small blood vessels of the arms that results from both photodamage as well as advancing age. This, coupled with the loss of subcutaneous fat and connective tissue, leaves the upper extremities more susceptible to the ill effects of even minor trauma. Post traumatic blood is extravasated into the surrounding dermis producing a dark blue-black discoloration of the skin, which is thought to be due to the iron laden protein hemosiderin.

This natural, age-related, bruising is exacerbated by the increased use of blood thinners, such as Warfin (Coumadin), Aspirin, Xerelto, Pradaxa, Eliquis, Savaysa, Plavix and Cortisteroids. With increased age, this bruising does not always resolve naturally and may progress to the formation of permanent hemosiderin staining. Following a bleed, extravasated red blood cells are deposited into the extravascular space and subsequently die, releasing hemoglobin, the oxygen carrying component of the blood.

Current treatments for Senile Purpura and prevention of hemosiderin staining may include over the counter creams and ointments or natural remedies. Natural remedies are unreliable at best. Over the counter creams and ointments tend to rely on anti-inflammatories, which may not reach the deep tissues involved in hemosiderin staining and may prove ineffective in treating Senile Purpura.

Thus, a topical formulation for treating bruising solving the aforementioned problems is desired.

### SUMMARY

A topical formulation for treating bruising can include an exfoliant, a protein denaturant, a penetration enhancer, triethanolamine, and water. In an embodiment the topical formulation may include one or more other components selected from a preservative, a detergent, a skin denaturant, and a base. The topical formulation can be particularly effective for reversing bruising caused by Senile Purpura and/or preventing hemosiderin staining.

The topical formulation may be combined with any delivery system known in the art, such as an ointment, cream, lotion, liquid, roll-on, patch, gel, paste, micelle, dermal patch, or an occlusion.

An embodiment of the present subject matter is directed to a method of treating bruising, including administering to a patient in need thereof at least one topical formulation according to the present subject matter. The method of treating bruising may include iontophoresis.

These and other features of the present disclosure will become readily apparent upon further review of the following specification and drawings.

### BRIEF DESCRIPTION OF THE DRAWINGS

FIG. 1A shows an arm bruise on day 1 before treatment with the topical formulation for treating bruising according to the present subject matter.

FIG. 1B shows the FIG. 1A bruise after three days of treatment with the topical formulation for treating bruising.

FIG. 1C shows the FIGS. 1A and 1B bruise after five days of treatment with the topical formulation for treating bruising.

FIG. 2A shows an arm bruise on day 1 before treatment with the topical formulation for treating bruising.

FIG. 2B shows the FIG. 2A bruise after three days of treatment with the topical formulation for treating bruising.

FIG. 2C shows the FIG. 2A and 2B bruise after six days of treatment with the topical formulation for treating bruising.

### DETAILED DESCRIPTION OF THE PREFERRED EMBODIMENTS

A topical formulation for treating bruising can be topically administered to a patient for treating bruising, and particularly, for treating bruising associated with purpura or for preventing hemosiderin staining. The composition can be directly applied to a bruise on the skin. The topical formulation for treating bruising can include an exfoliant, a protein denaturant, a penetration enhancer, triethanolamine, and water. Optionally, the topical formulation can include a humectant, e.g., glycerin, and/or a scented natural oil, e.g., lavender or rosemary. The humectant can mitigate the drying effects of the alcohols in the formulation. The scented oil can provide fragrance as well as reduce the evaporation rate of volatiles.

As used herein to define a weight by volume percentage, the term “about” shall mean within 1.0% of the specified weight by volume percentage.

The exfoliant may be any exfoliant known in the art, including but not limited to citric acid, salicylic acid, glycolic acid, malic acid, tartaric acid, or mechanical exfoliants.

The protein denaturant may be any protein denaturant known in the art, including but not limited to Butanol, pentanol, octanol, other alcohols, or urea.

The penetration enhancer may be any penetration enhancer known in the art, including but not limited to dimethyl isosorbide (DMI), dimethyl sulfoxide, an azone such as laurocapram, a pyrrolidone, an alcohol, an alkanoid such as ethanol or decanol, glycols, an essential oil, a mineral oil, a vegetable oil, a terpene, a terpenoid, an oxazolidinone, or urea.

In an embodiment the exfoliant may be citric acid, the protein denaturant may be butanol (2-butanol), and the penetration enhancer may be DMI. The citric acid may range from about 1.0% to about 10.0% by weight of the topical formulation (e.g., 0.3% to 8% by weight or 1% by weight). The butanol may range from about 0.1% to about 10.0% by weight of the topical formulation. The DMI may range from about 0.5% to about 15.0% by weight of the topical formulation. The water may range from about 5.0% to about 68.0% by weight of the topical formulation. TEA may be added to the formulation in an amount sufficient to adjust the final solution to a pH ranging from about pH 3.8 to about pH 4.2, e.g., pH 4.0.

In an embodiment the topical formulation may include one or more other components selected from a preservative, a detergent, a skin denaturant, and a base.

The preservative may be any preservative known in the art, including but not limited to benzalkonium chloride (BKC) or Germall® Plus, a preservative including 60% by weight propylene glycol, 39.6% by weight diazolidinyl urea (DU) and 0.4% iodopropynyl butylcarbamate (IPBC).

The detergent may be any detergent known in the art, including but not limited to sodium laureth sulfate (SLES), sodium laurel sulfate, ammonium lauryl sulfate, sodium pareth sulfate, magnesiumlaureth sulfate, or an alkylbenzene sulfonate.

The skin denaturant may be any skin denaturant known in the art, including but not limited to isopropyl alcohol (IPA), a fatty ester, a fatty alcohol, isopropyl mystirate, methanol, or ethanol.

In an embodiment, the detergent may be SLES ranging from about 0.01% to about 10.0% by weight of the topical formulation.

In an embodiment, the skin denaturant may be isopropyl alcohol (IPA). The IPA may range from about 1.0% by weight to about 70.0% by weight of the topical formulation.

In an embodiment, the topical formulation may include at least one preservative. For example, the topical formulation may include about 0.5% by weight Germall® Plus, or about 0.5% by weight BKC.

The composition can be directly administered to a bruised site on the skin of the patient. Once applied on the skin, it is preferable to apply a bandage or other suitable dressing over the composition for occlusion. The dressing can be sterile or non-sterile. A particular formulation of the present composition selected for treatment may be based upon a patient's skin sensitivity classification. The administration may include iontophoresis. It should be understood that gamma radiation of a dressing including the composition may be employed instead of or in addition to including a preservative in the composition.

In an embodiment, the composition can include a formulation suitable for most patients, regardless of their skin sensitivity classification, herein “general skin formulation.”

A first embodiment of the general skin formulation may include about 1.0% by weight citric acid, about 0.3% by weight butanol, about 30.0% by weight IPA, about 10.0% by weight DMI, and about 0.5% by weight of a preservative, preferably Germall® Plus. The composition may be adjusted to a pH of about 4.0 by adding TEA, e.g., about 0.40% by weight TEA. A remainder of the topical composition may be water. The general formulation can be applied to the skin and occluded, as described previously.

A second embodiment of the general skin formulation may include about 1.0% by weight citric acid, about 0.2% by weight butanol, about 5.0% by weight IPA, about 10.0% by weight DMI, and about 0.5% by weight of a preservative, preferably Germall® Plus. The composition may be adjusted to a pH of about 4.0 by adding TEA, e.g., about 0.40% by weight TEA. A remainder of the topical composition may be water. The general formulation can be applied to the skin and occluded, as described previously.

In an embodiment, the composition can include a formulation particularly suitable for patients suffering from sensitive skin, herein “sensitive skin formulation.” Patients identified as having “sensitive skin,” according to the present teachings, typically have skin that is not very dry and generally smooth to the touch. These patients generally tolerate most topical compositions, but may have a history of occasional negative reactions to topical products containing harsh sensitizing chemicals. These patients typically do not suffer from itchy skin.

A first embodiment of the sensitive skin formulation may include about 1.0% by weight citric acid, about 0.5% by weight 2-butanol, about 0.5% by weight SLES, about 20.0% by weight IPA, about 10.0% by weight DMI, and about 0.5% by weight of a preservative, preferably Germall® Plus. The composition may be adjusted to a pH of about 4.0 using TEA. A remainder of the topical composition may be water. Preferably, this embodiment of the sensitive skin formulation is used with a non-sterile dressing.

A second embodiment of the sensitive skin formulation may include about 1.0% by weight citric acid, about 0.5% by weight 2-butanol, about 0.5% by weight SLES, about 20.0% by weight IPA, and about 10.0% by weight DMI. This composition may be adjusted to a pH of about 4.0 using TEA. A remainder of the topical composition may be water. Preferably, this embodiment of the sensitive skin formulation is used with a sterile dressing.

In an embodiment, the composition can include a formulation particularly suitable for patients suffering from non-sensitive skin, herein “non-sensitive skin formulation.” Patients identified as having “non-sensitive skin,” according to the present teachings, typically have normal, well-hydrated, smooth skin that is soft and smooth to touch. These patients typically do not suffer from itchy or flaky skin.

A first embodiment of the non-sensitive skin formulation may include about 10.0% by weight citric acid, about 10.0% by weight 2-butanol, about 1.0% by weight SLES, and about 15.0% by weight DMI. This composition may be adjusted to a pH of about 4.0 using TEA. A remainder of the topical composition may be water. Preferably, this embodiment of the non-sensitive skin formulation is used with a sterile dressing.

A second embodiment of the non-sensitive skin formulation may include about 10.0% by weight citric acid, about 10.0% by weight 2-butanol, about 1.0% by weight SLES, about 15.0% by weight DMI, and about 0.5% by weight of a preservative, preferably German( )Plus. This composition may be adjusted to a pH of about 4.0 using TEA. A remainder of the topical composition may be water. Preferably, this embodiment of the non-sensitive skin formulation is used with a non-sterile dressing.

In an embodiment, the composition can include a formulation particularly suitable for patients suffering from very sensitive skin, herein “very sensitive skin formulation.” Patients identified as having “very sensitive skin,” according to the present teachings, typically have dry, flaky, itchy skin, with patches of redness. These patients generally have a history of sensitivity to topical products, such as soaps, make-up removers, and other topical skin care products.

A first embodiment of the very sensitive skin formulation may include about 1.0% by weight citric acid, about 0.2% by weight 2-butanol, about 20.0% by weight IPA, about 10.0% by weight DMI, and about 0.5% by weight of a preservative, preferably Germall® Plus. This composition may be adjusted to a pH of about 4.0 using TEA. A remainder of the topical composition may be water. Preferably, this embodiment of the very sensitive skin formulation is used with a non-sterile dressing.

A second embodiment of the very sensitive skin formulation may include about 1.0% by weight citric acid, about 0.2% by weight 2-butanol, about 20.0% by weight IPA, and about 10.0% by weight DMI. This composition may be adjusted to a pH of about 4.0 using TEA. A remainder of the topical composition may be water. Preferably, this embodiment of the very sensitive skin formulation is used with a sterile dressing.

A third embodiment of the very sensitive skin formulation may be in ointment forms and may include about 1.0% by weight citric acid, about 0.2% by weight 2-butanol, about 20.0% by weight IPA, about 10.0% by weight DMI, about 0.5% by weight of a preservative, preferably Germall® Plus, and about 5.0% by weight water. This composition may be adjusted to a pH of about 4.0 using TEA. A remainder of the topical composition may be an appropriate base composition. The appropriate base composition may be any base ointment suitable for delivering an ointment.

In an embodiment, the base ointment may be AquaBASE Ointment. AquaBASE Ointment may include petrolatum, mineral oil, mineral wax, wool wax alcohol, and cholesterol. Preferably, this embodiment of the very sensitive skin formulation is used with a non-sterile dressing.

According to an embodiment, a method of treating bruising or treating or preventing a disease associated with bruising can include administering to a patient in need thereof at least one topical formulation disclosed herein.

In an embodiment, diseases associated with bruising can include at least one of Senile Purpura and hemosiderin staining.

An embodiment of the present subject matter is directed to a method of treating a bruise, comprising administering to a patient in need thereof a therapeutically effective amount of topical formulation.

The topical formulation may be used with any known delivery system, such as an ointment, cream, lotion, liquid, roll-on, patch, gel, paste, micelle, dermal patch, or an occlusion. Occlusion can be achieved by applying a liquid form of the topical formulation to a dressing, such as an adhesive gauze pad or strip, to provide a medicated dressing and then applying the medicated dressing to the bruise. In an embodiment, the composition may be an ointment and the occlusion may be a cotton free adhesive layer. The topical formulation may further include one or more penetration enhancers, denaturants, pH regulators, exfoliates, stratum corneum penetrators, and/or keratolytic agents.

In an embodiment, the topical formulation, e.g., preservative-free formulations, may be applied to a dressing, securely wrapped, and gamma irradiated prior to use.

In an embodiment, a topical formulation for use in treating very sensitive skin may instead be applied continuously to a patient having sensitive or non-sensitive skin. The treatment time may be extended to achieve bruise reduction.

In an embodiment, the topical formulation for treating bruising may be prepared by mixing the preservative with water to form a first solution, adding citric acid to the first solution to form a second solution, adding butanol to the second solution to form a third solution, adding DMI to the third solution to form a fourth solution, and adding IPA to the fourth solution to form a fifth solution. An appropriate amount of TEA can be added to the fifth solution to provide a titrated solution having a pH ranging from about 3.8 to about 4.2. Water may then be added to the titrated solution to provide the final formulation.

In an alternative embodiment, the appropriate amount of TEA can be added to the fifth solution to provide a titrated solution having a pH of about 4.0.

In an alternative embodiment, the topical formulation for treating bruising may be prepared by mixing citric acid with water to create a first solution. IPA and DMI may be mixed to create a second solution. Optionally, 2-butanol and/or SLES can be mixed into the second solution. The first solution and the second solution may then be mixed thoroughly, creating a third solution. If desired, BKC may then be mixed into the third solution. The pH of the third solution may then be adjusted to a pH ranging from about pH 3.8 to about 4.2, e.g., 4.0, by dropwise addition of TEA. The weight of the solution may then be raised to about 94.0% by addition of an appropriate base. For a liquid formulation, the appropriate base may be water. For an ointment, the appropriate base may be a suitable ointment. As a final step, the pH may once again be checked, and if necessary, TEA may be added to arrive at a final pH of about 3.8 to about 4.2. Optionally, the final pH may be about 4.0.

### EXAMPLE 1

### Testing of Sterile Topical Formulations for Sensitive and Normal Skin

Patients suffering from purpura were selected and their skin sensitivity was assessed. Each patient's purpura, or bruises, were categorized based on size. A small bruise was between about 1 and 2 cm in diameter. A medium bruise was between about 2 and 4 cm in diameter. A large bruise was between about 4 cm and about 8 cm in in diameter. The sterile topical formulations for patients with sensitive and normal skin were used as appropriate for each patient. An amount ranging from about 6 to about 8 drops of the topical formulation was applied to a small bruise. An amount ranging from about 12 to about 16 drops of the topical formulation was applied to a medium bruise, and an amount ranging from about 20 to about 24 drops of the topical formulation was applied to a large bruise. The topical formulation was first applied to a dressing, which was then placed over the bruised area of the skin. The occlusion provided by the dressing may be improved by treating (wrapping) the dressing with Coban. The dressing was left occluding the skin for about 8 to 12 hours. The dressing was re-applied daily until the purpura was at least 90% resolved. This test demonstrated that the topical formulation was effective at resolving purpura within two to five days of commencing treatment (Table 1).

TABLE 1
In Vivo Testing of Topical Formulation

| | | Days to 90% | | |
|---|---|---|---|---|
| Age | Sex | Resolution | History | Medications |
| 84 | F | 2 | N/A | ASA |
| 78 | F | 5 | N/A | N/A |
| 72 | M | 4 | Atrial Fibrillation | Xeralto & ASA |
| 84 | F | 5 | N/A | N/A |
| 71 | M | 4 | Cardiac Stent | Plavix |

### EXAMPLE 2

### Testing of Sterile Topical Formulations for Sensitive, Non-Sensitive, and Very Sensitive Skin

The general procedure of Example 1 was repeated, but modified to address patients' skin sensitivity. When treating patients with very sensitive skin, the ointment including the topical formulation was applied directly to the skin, gently rubbed for 10 to 15 seconds, and covered by an adhesive dressing with the cotton pad removed, allowing for direct contact between the ointment and the plastic dressing. The dressing was left in place for 12 hours, then removed. The ointment was reapplied with gentle rubbing and without any occlusion. The ointment was applied for four hours up to three times daily, and treatment was continued until either bruise resolution (defined as at least 90% reduction in bruise size) or the appearance of substantial side effects that interfered with continuation of treatment. This treatment generally lasted less than six days. Exemplary data from this test are provided in Table 2.

TABLE 2
Selective Test Results

| Age | Sex | Skin Type | Contact Time to 90% resolution |
|---|---|---|---|
| 77 | F | Non-Sensitive | 32.5 Hours |
| 71 | M | Sensitive | 32.75 Hours |
| 84 | F | Non-Sensitive | 28.33 Hours |
| 72 | M | Sensitive | 42.75 Hours |
| 70 | M | Very Sensitive | 48.5 Hours |
| 66 | M | Very Sensitive | 52.25 Hours |

### EXAMPLE 3

### Test Results

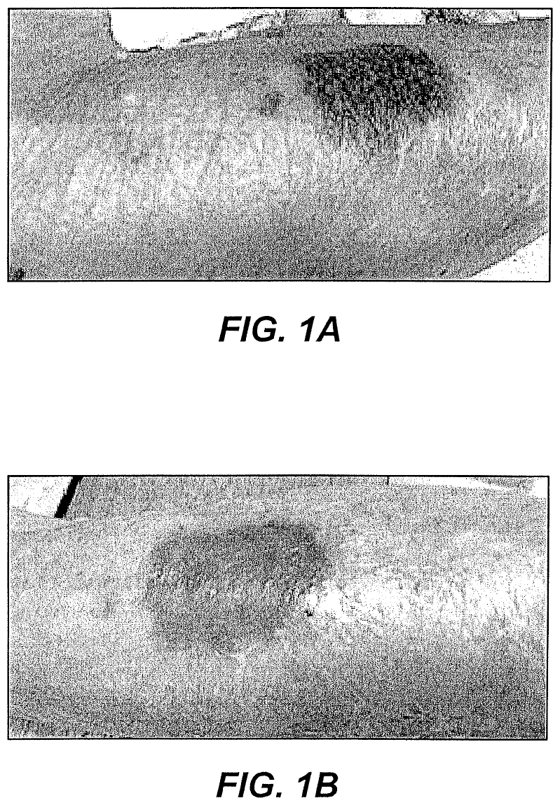

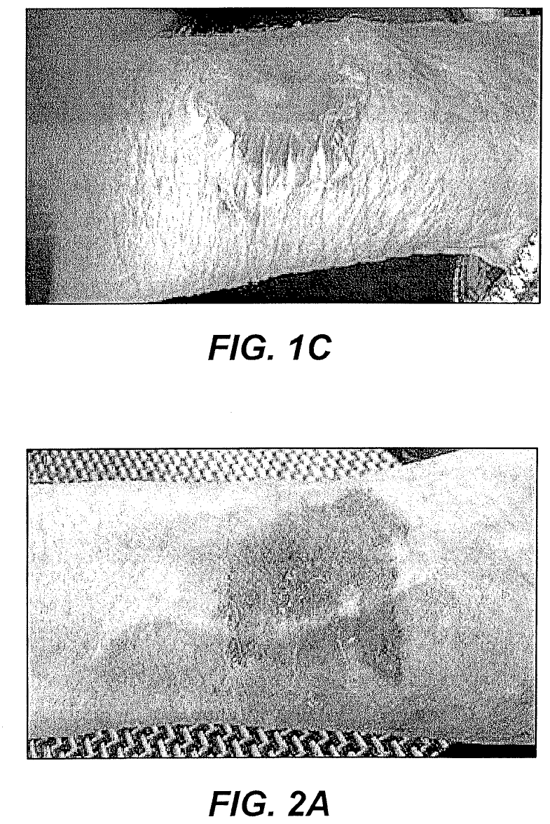

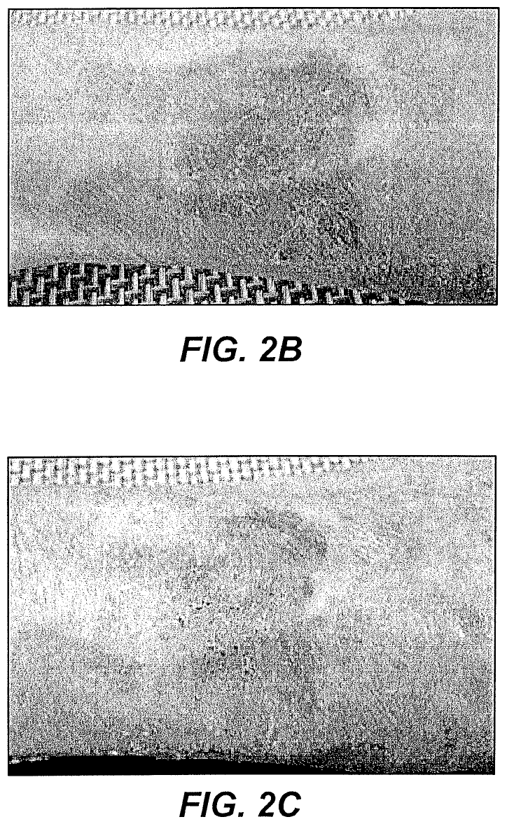

The procedure described in Example 1 was performed on two test subjects, each with a bruise on their left forearm. The bruising present pre-treatment is shown in FIGS. 1A and 2A. The reduction of bruising after three days of treatment with a topical formulation for treating bruising may be seen in FIGS. 1B and 2B. Significant resolution of bruising after five days and six days, respectively, is shown in FIGS. 1C and 2C.

### EXAMPLE 4

### Testing of General Formulations

Three patients suffering from purpura skin were treated with the first embodiment of the general formulation. For this test, patient skin sensitivity prior to treatment was not considered. Patients with bruising on the arm received 1 cc of the first embodiment of the general formulation. A patient with a small bruise on the hand received 0.5 cc of the first embodiment of the general formulation. The general formulation was first applied to a dressing, which was then placed over the bruised area of the skin. The bruising was about 90% resolved or healed within a time period ranging from about 35 hours to about 52 hours.

A patient suffering from purpura skin was treated with the second embodiment of the general formulation. For this test, patient skin sensitivity prior to treatment was not considered. The patient received 0.5 cc of the second embodiment of the general formulation. The general formulation was first applied to a dressing, which was then placed over the bruised area of the skin. The bruising was about 90% resolved or healed within about 112 hours. The results are summarized in Table 3 below.

TABLE 3

| | | Contact Time | | |
|---|---|---|---|---|
| | | to 90% | | |
| Age | Sex | resolution | Formula | Profile |
| 71 | M | 45 Hours | General | taking Xeralto, blood pressure |
| | | | | medication and |
| | | | | cardiac medication |
| 67 | M | 35 Hours | General | taking blood thinners and blood |
| | | | | pressure medication |
| 52 | M | 52 Hours | General | taking diabetes medication and |
| | | | | ASA for cardiac stent |
| 60 | F | 112 Hours | Alternative | taking Plavix and blood |
| | | | | pressure medication |

In another embodiment, a topical formulation for treating bruising can be topically administered to a patient for treating bruising, and particularly, for treating bruising associated with purpura or for preventing hemosiderin staining. The composition can be directly applied to a bruise on the skin. The topical formulation for treating bruising can include a protein denaturant, a penetration enhancer, and water. It is noted that the triethanolamine (TEA) or citric acid discussed in the other formulations may be included in the new formulation for pH adjustments between 5.5 and 6.5 on an “as needed” basis. The preferred formulation is based on an understanding of the nature of bruising. Specifically, the hemoglobulin molecule consists of iron and the protein globulin protein, and together carry oxygen throughout the body. Very simply, a bruise is trapped hemoglobulin under the skin. Iron, the source of the dark color associated with bruising is housed in the hemoglobin and must be released by uncoiling the globulin protein, a process known as denaturation. Although this process occurs naturally over time, the addition of the protein denaturant 2-butanol to the formulation accelerates this process. Once released, the iron molecules are removed through existing disposal channels.

Organic alcohols are known to have protein denaturant properties and their denaturant activity generally increases with chain length. For example, Methanol, Propanol, Ethanol, Pentanol, Hexanol, Urea, Guanidinium chloride, Lithium perchlorate, Sodium dodecyl sulfate are protein denaturants that show efficacy in bruise resolution. However, the 4-carbon alcohol 2-Butanol was selected because of its superior denaturant properties along with its long history of safety in the cosmetic industry.

The 2-Butanol concentrations from 2% to 20% by weight of the topical formulation demonstrated varying degrees of efficacy and side-effects. At the lower concentrations the frequency and severity of side-effects decreased, as did the efficacy of bruise resolution. At the higher concentrations the frequency and severity of side-effects, such as pain, itching, redness and exfoliation increased. The side-effects were resolved at 4% -12%, with 10% being the preferred concentration since it provided very good bruise resolution. The 2-Butanol alone in the topical formulation would not provide the bruise resolution.

The 2-Butanol needs a specific penetrant in the topical formulation. Skin penetration enhancers are known in the art. For example, sulfoxides, azones, pyrrolidone and the like have been used. However, the combination of dimethyl isosorbide (DMI) and propylene glycol acts as a synergistic penetrant for bruise resolution without side effects. The 2-butanol acts as both a protein denaturant as well as a penetration enhancer and assists the combined effects of the pentrant. Thus, the combined activity of dimethyl isosorbide (DMI), propylene glycol and 2-butanol facilitates the movement of the 2-butanol through the multiple skin layers on its way to the dermis, where it exercises its protein denaturant activity on the globulin molecule. The topical formulation uses 20-40% DMI along with 1-10% propylene glycol as the preferred penetrant; the preferred amounts being 30% and 5%, respectively. The synergy between DMI and propylene glycol facilitates the efficient movement of 2-Butanol to the dermis, the site of bruising.

Poloxamer 407 is the preferred poloxamer used for its surfactant properties. Specifically, it is used for dissolving oily ingredients in water. At 16% -19% of the topical formulation, poloxamer 407 is a liquid at room temperature that becomes a gel at normal body temperature (97° F.-99 ° F.). The preferred concentration is 18% thereby overcoming any failure to gel or remaining a liquid at normal skin temperature. Sodium chloride: was added to increase the poloxamer 407 gel strength.

The efficacy of preservatives, acting alone or in combination, are well documented in the literature. Although Germall® Plus is the preferred preservative at 0.1-0.5%, other preservatives, such as parabens and isothiazolinones, acting singularly or in combination could provide an acceptable preservative activity.

Topical formulation by weight %:

| | |
|---|---|
| Poloxamer 407 | 16-19 |
| Dimethyl isosorbide | 20-40 |
| 2-Butanol | 4-12 |
| Propylene glycol | 1-10 |
| Sodium Chloride | 0.1-0.5 |
| Germall ® Plus | 0.1-0.5 |
| Water | Balance |

It is to be understood that the topical formulation for treating bruising is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A topical formulation for treating bruising, the formulation comprising a protein denaturant, a penetration enhancer, a preservative, and water.

2. The topical formulation for treating bruising according to claim 1, wherein the protein denaturant is 2-Butanol and the penetration enhancer is dimethyl isosorbide and propylene glycol.

3. The topical formulation for treating bruising according to claim 2, wherein the 2-Butanol is 4-12% weight of the formulation, the dimethyl isosorbide is 20-40% and the propylene glycol is 1-10%.

4. The topical formulation for treating bruising according to claim 3, wherein the 2-Butanol is 10% weight of the formulation, the dimethyl isosorbide is 30% and the propylene glycol is 5%.

5. A topical formulation for treating bruising, the formulation comprising poloxamer 407, dimethyl isosorbide, 2-Butanol, propylene glycol, Sodium Chloride, Germall® Plus, and water.

6. A topical formulation for treating bruising, comprising the formulation by % weight:

| | |
|---|---|
| Poloxamer 407 | 16-19 |
| Dimethyl isosorbide | 20-40 |
| 2-Butanol | 4-12 |
| Propylene glycol | 1-10 |
| Sodium Chloride | 0.1-0.5 |
| Germall ® Plus | 0.1-0.5 |
| Water | Balance |

7. The topical formulation for treating bruising according to claim 6, wherein the formulation includes dimethyl isosorbide at 30%, 2-Butanol at 10%, and propylene glycol at 5%.

8. The topical formulation for treating bruising according to claim 6, wherein the formulation includes dimethyl isosorbide at 30%.

9. The topical formulation for treating bruising according to claim 6, wherein the formulation includes 2-Butanol at 10%.

10. The topical formulation for treating bruising according to claim 6, wherein the formulation includes propylene glycol at 5%.

11. The topical formulation for treating bruising according to claim 6, wherein the formulation is in a topical administration form selected from the group consisting of an ointment, a ointment cream, a lotion, a thermogallin liquid, a roll-on, a patch, a gel, a paste, a micelle, a dermal patch, or an occlusion.

12. A method of treating bruising, comprising administering the topical formulation of claim 6 to a patient in need thereof.
